(19) United States
(12) Patent Application Publication
(10) Pub. No.: US 2025/0345567 A1
(43) Pub. Date: Nov. 13, 2025

(54) Catheter Assembly Near Port Connector with Improved Backpressure Performance and Leak Prevention

(71) Applicant: Becton, Dickinson and Company, Franklin Lakes, NJ (US)

(72) Inventors: Erica E. Neumann, Sandy, UT (US); Weston F. Harding, Lehi, UT (US); Alison Nicole Anderson, Salt Lake City, UT (US); Thomas A. Solosko, Draper, UT (US); Shaun Staley, Murray, UT (US); Jonathan Karl Burkholz, Salt Lake City, UT (US); Curtis H. Blanchard, Herriman, UT (US); Ralph L. Sonderegger, Farmington, UT (US); Michael Glen Beeman, Salt Lake City, UT (US); Dorien Gemma Butter, Millcreek, UT (US); Chris Zollinger, Chino, CA (US)

(21) Appl. No.: 19/202,385

(22) Filed: May 8, 2025

Related U.S. Application Data
(60) Provisional application No. 63/644,693, filed on May 9, 2024.

(51) Int. Cl. A61M 25/06; A61M 39/02; A61M 39/06; A61M 39/16; A61M 39/20; A61M 39/24

(52) U.S. Cl. CPC A61M 25/0606; A61M 39/0613; A61M 39/16; A61M 39/20; A61M 39/24; A61M 2039/0202; A61M 2039/0205; A61M 2039/062; A61M 2039/0646; A61M 2039/066; A61M 2039/244; A61M 2039/248; A61M 2205/0216; A61M 2205/3331

(57) ABSTRACT

A catheter assembly is provided that includes a catheter, a catheter adapter, a near port connector connected to the catheter adapter port, and an extension set coupled to the near port connector and including a far port connector. The near port connector includes a distal port, a proximal port, and a side port, with the extension set connected to the side port and a split-septum valve provided in the proximal port. The near port connector may be configured to withstand a proximally-directed fluid backpressure of greater than 30 psi to prevent fluid leaks during a fluid injection through the far port connector, or may have a fluid path shut-off attached thereto to selectively close a fluid path through the near port connector and the split-septum valve, to prevent fluid leaks out from the near port connector during a fluid injection through the far port connector.

### CROSS-REFERENCE TO RELATED APPLICATION

The present application claims priority to U.S. Provisional Patent Application No. 63/644,693, entitled “Catheter Assembly Near Port Connector with Improved Backpressure Performance and Leak Prevention” filed May 9, 2024, the entire disclosure of which is hereby incorporated by reference in its entirety.

### BACKGROUND OF THE INVENTION

### Field of the Invention

The present disclosure relates generally to a catheter assembly and, in particular, to a catheter assembly that includes a near port connector having improved backpressure performance and leak prevention.

### Background of the Invention

Vascular access devices (VADs) are used in the medical field to access peripheral vasculature of a patient for purposes of infusion therapy and/or blood withdrawal. Common types of VADs include over-the-needle peripheral intravenous catheters (PIVCs), peripherally inserted central catheters (PICCs), central venous catheters (CVCs), and midline catheters. The VAD may be indwelling for short term (days), moderate term (weeks), or long term (months to years).

In some instances, the VAD is part of a larger catheter system or assembly, where tubing extends out from an adapter or connector of the VAD to provide additional flexibility for medical professionals in terms of administering and/or withdrawing fluids from the VAD—with such tubing being part of an extension set that is integrated with the VAD (an integrated catheter system) or provided separate from the VAD (a non-integrated catheter system), according to various embodiments. Examples of existing integrated catheter systems from Becton, Dickinson and Company (“BD”) include the BD Nexiva™ closed peripheral IV catheter system, the BD Nexiva™ Diffusics™ closed IV catheter system, and the BD Nexiva™ NearPort™ closed peripheral IV catheter system, as non-limiting examples.

Catheter systems such as those mentioned above may often include multiple connectors or access ports by which external devices may be connected to the catheter system, in order to provide for the administering and/or withdrawing fluids from the VAD. Such connectors may generally be characterized as a “near port” connector and a “far port” connector. The near port connector may be ideal for connection of a blood draw device such as the PIVO™ from Becton, Dickinson and Company, which is configured to advance a flexible, internal probe or flow tube into/through the indwelling catheter of the catheter system. The far port connector may be ideal for connection of a fluid delivery device thereto, for an intravenous infusion via a gravity flow drip, syringe push, or saline flush, as non-limiting examples. When utilizing the far port for these applications, it is known that a backpressure may be applied to a split septum valve of the near port connector. The near port connector is typically rated for ˜30 psi of backpressure, so that in most instances it is able to withstand the backpressure applied thereto during fluid delivery through the far port. However, in some instances-such as when the indwelling catheter is occluded—it is possible for the backpressure to exceed the ˜30 psi limit of the near port connector, and this may cause fluid to flow proximally out through the split septum valve, so that a leak occurs out from the near port connector.

Accordingly, a need exists for catheter systems that include a near port connector that exhibits improved backpressure performance. The near port connector should be configured to withstand backpressures of greater than 30 psi, in order to prevent leakage from the connector in a high pressure environment.

### SUMMARY OF THE INVENTION

Provided herein is a catheter assembly that includes a catheter comprising a catheter distal end and a catheter proximal end, and a catheter adapter comprising an adapter body including a distal adapter port, a proximal adapter port, and a side adapter port, the adapter body defining a lumen therein extending between the distal adapter port and the proximal adapter port, wherein the catheter proximal end is secured to the distal adapter port, and wherein the side adapter port is in fluid communication with the lumen. The catheter assembly also includes a first fluid conduit comprising a distal end and a proximal end, the distal end of the first fluid conduit connected to the proximal adapter port, a near port connector comprising a distal connector port, a proximal connector port, and a side connector port, the distal connector port connected to the proximal end of the first fluid conduit, with the near port connector comprising a split-septum valve provided in the proximal connector port, and an extension set coupled to the side connector port, the extension set comprising a second fluid conduit comprising a distal end and a proximal end, the distal end coupled to the side connector port, and a far port connector provided at the proximal end of the second fluid conduit. The near port connector is configured to withstand a proximally-directed fluid backpressure of greater than 30 pounds-per-square inch (psi), so as to prevent fluid leaks out from the near port connector during a fluid injection through the far port connector.

In some embodiments, the near port connector comprises a duckbill valve positioned within a lumen of the near port connector, proximal to the side connector port and distal to the split-septum valve, the duckbill valve having a higher backpressure rating than the split-septum valve.

In some embodiments, the near port connector comprises a secondary valve positioned within a lumen of the near port connector, proximal to the side connector port and distal to the split-septum valve, the secondary valve having a higher backpressure rating than the split-septum valve, and a valve actuator element positioned within a lumen of the near port connector and proximally from the secondary valve, the valve actuator element including a channel formed therethrough, wherein the valve actuator element is moveable distally within the lumen responsive to introduction of a male connector into the split-septum valve, with the valve actuator element opening the secondary valve upon such distal movement.

In some embodiments, the near port connector comprises a swinging hinge valve positioned within a lumen of the near port connector, proximal to the side connector port and distal to the split-septum valve, the swinging hinge valve having a higher backpressure rating than the split-septum valve.

In some embodiments, the split-septum valve comprises a septum membrane having a slit formed therethrough and a sidewall extending distally from the septum membrane, with the septum membrane and sidewall defining a cavity in the split-septum valve.

In some embodiments, an underside of the septum membrane has a duckbill shape.

In some embodiments, an underside of the septum membrane has a dome shape.

In some embodiments, the split-septum valve comprises a bi-material component, with the sidewall formed of a first material and the septum membrane formed of a second material that has a higher modulus of elasticity than the first material.

In some embodiments, the near port connector comprises a distal housing portion and a proximal housing portion that engage each other to secure the split-septum valve within the near port connector.

In some embodiments, the sidewall comprises a pair of protrusions on opposing sides thereof, with the pair of protrusions extending radially inward into the cavity to narrow the cavity, with the pair of protrusions configured to deflect inwardly toward each other and come in contact with each other upon application of a radially inward-directed pressure thereto, thereby forming a secondary seal in the split-septum valve having a higher backpressure rating than the septum membrane.

In some embodiments, the proximal housing portion includes a reduced diameter portion formed by a pair of radially-inward extending bumps, with the pair of radially-inward extending bumps pressing inwardly against the split-septum valve in an area of the pair of protrusions when the distal housing portion is secured to the proximal housing portion, thereby deflecting the pair of protrusions to form the secondary seal.

In some embodiments, the proximal housing portion comprises a housing shelf and the sidewall comprises a valve shelf, with the housing shelf engaging with the valve shelf to increase a contact surface area between the split-septum valve and the proximal housing portion, in order to prevent proximal movement of the split-septum valve within the proximal housing portion responsive to a proximally-directed fluid backpressure.

In some embodiments, a thickness of the sidewall is increased or decreased, so as to decrease or increase a compliance of the split-septum valve, respectively, so as to increase the backpressure rating of the split-septum valve.

In some embodiments, the thickness of the sidewall is decreased, with the sidewall of the split septum valve having a rectangular cut-out on an outer surface thereof, a curved cut-out on the outer surface thereof, curved cut-outs on inner and outer surfaces thereof, or an accordion-like profile with material removed from inner and outer surfaces thereof.

In some embodiments, the proximal housing portion comprises an inner surface defining a compressive zone that engages the split-septum valve to maintain the split-septum valve in a closed configuration, and wherein the inner surface in the compressive zone has a taper angle of between 25 and 45 degrees.

In some embodiments, the inner surface in the compressive zone comprises a proximal-most segment and a distal-most segment having different taper angles, with the taper angle of the proximal-most segment being larger than the taper angle of the distal-most segment.

In some embodiments, the near port connector is configured to withstand a proximally-directed fluid backpressure of up to 60-80 psi, and preferably up to 325 psi.

Also provided herein is a catheter assembly that includes a catheter comprising a catheter distal end and a catheter proximal end, and a catheter adapter comprising an adapter body including a distal adapter port, a proximal adapter port, and a side adapter port, the adapter body defining a lumen therein extending between the distal adapter port and the proximal adapter port, wherein the catheter proximal end is secured to the distal adapter port, and wherein the side adapter port is in fluid communication with the lumen. The catheter assembly also includes a first fluid conduit comprising a distal end and a proximal end, the distal end of the first fluid conduit connected to the proximal adapter port, and a near port connector comprising a distal connector port, a proximal connector port, and a side connector port, the distal connector port connected to the proximal end of the first fluid conduit, with the near port connector comprising a split-septum valve provided in the proximal connector port. The catheter assembly further includes an extension set coupled to the side connector port, the extension set comprising a second fluid conduit comprising a distal end and a proximal end, the distal end coupled to the side connector port, and a far port connector provided at the proximal end of the second fluid conduit. The catheter assembly still further includes a fluid path shut-off attached to or integrated with the near port connector, to selectively close a fluid path through the near port connector and the split-septum valve, so as to prevent fluid leaks out from the near port connector during a fluid injection through the far port connector.

In some embodiments, the fluid path shut-off comprises an end cap coupled to the proximal connector port, with the end cap sealing off the split-septum valve.

In some embodiments, the end cap comprises a disinfecting foam pad retained within a cavity thereof, with the foam pad being compressed upon engaging of the end cap with the proximal connector port, so as to seal and disinfect the split-septum valve.

In some embodiments, the end cap comprises a tethered end cap including a cap member that couples to the proximal connector port to seal off the split-septum valve, a coupling loop configured to engage with the near port connector, and a tether connected the cap member and the coupling loop.

In some embodiments, the fluid path shut-off comprises a Tuohy-Borst valve that is integrated into the near port connector so as to be adjacent the split septum valve or integrated with the split septum valve, the Tuohy-Borst valve operable to close the fluid path through the near port connector and the split-septum valve.

In some embodiments, the fluid path shut-off comprises a ball valve that is integrated into the near port connector so as to be distal to the split septum valve and proximal from the side connector port, the ball valve operable to close the fluid path through the near port connector and the split-septum valve.

In some embodiments, the fluid path shut-off comprises a push-pull valve that is integrated into the near port connector so as to be distal to the split septum valve and proximal from the side connector port, the push-pull valve operable to close the fluid path through the near port connector and the split-septum valve.

In some embodiments, the fluid path shut-off comprises one of an in-line needle free connector, an anti-reflux or bi-direction valve, or an interlink type connector, that is coupled to the near port connector at either the distal connector port or the proximal connector port.

### BRIEF DESCRIPTION OF THE DRAWINGS

FIG. 1 is a top view of a catheter assembly, in accordance with an aspect of the disclosure;

FIG. 2 is a cross-sectional view of a near port connector included in the catheter assembly of FIG. 1, in accordance with an aspect of the disclosure;

FIG. 3 is a cross-sectional view of a near port connector included in the catheter assembly of FIG. 1, in accordance with an aspect of the disclosure;

FIG. 4 is a cross-sectional view of a near port connector included in the catheter assembly of FIG. 1, in accordance with an aspect of the disclosure;

FIG. 5 is a cross-sectional view of a near port connector included in the catheter assembly of FIG. 1, in accordance with an aspect of the disclosure;

FIG. 6A is a cross-sectional view of a near port connector included in the catheter assembly of FIG. 1, in accordance with an aspect of the disclosure;

FIG. 6B is a cross-sectional view of a near port connector included in the catheter assembly of FIG. 1, in accordance with an aspect of the disclosure;

FIG. 6C is a cross-sectional view of a near port connector included in the catheter assembly of FIG. 1, in accordance with an aspect of the disclosure;

FIG. 6D is a cross-sectional view of a near port connector included in the catheter assembly of FIG. 1, in accordance with an aspect of the disclosure;

FIG. 7A is a cross-sectional view of a near port connector included in the catheter assembly of FIG. 1, in accordance with an aspect of the disclosure;

FIG. 7B is a cross-sectional view of a near port connector included in the catheter assembly of FIG. 1, in accordance with an aspect of the disclosure;

FIG. 8 is a cross-sectional view of a near port connector included in the catheter assembly of FIG. 1, in accordance with an aspect of the disclosure;

FIG. 9 is a cross-sectional view of a near port connector included in the catheter assembly of FIG. 1, in accordance with an aspect of the disclosure;

FIG. 10 is a cross-sectional view of a near port connector included in the catheter assembly of FIG. 1, in accordance with an aspect of the disclosure;

FIG. 11 is a cross-sectional view of a near port connector included in the catheter assembly of FIG. 1, in accordance with an aspect of the disclosure;

FIG. 12 is a cross-sectional view of a near port connector included in the catheter assembly of FIG. 1, in accordance with an aspect of the disclosure;

FIG. 13A is a cross-sectional view of a near port connector included in the catheter assembly of FIG. 1, in accordance with an aspect of the disclosure;

FIG. 13B is a cross-sectional view of a near port connector included in the catheter assembly of FIG. 1, in accordance with an aspect of the disclosure;

FIG. 13C is a cross-sectional view of a near port connector included in the catheter assembly of FIG. 1, in accordance with an aspect of the disclosure;

FIG. 13D is a cross-sectional view of a near port connector included in the catheter assembly of FIG. 1, in accordance with an aspect of the disclosure;

FIG. 14 is a perspective view of an end cap useable with the near port connector of FIG. 2, in accordance with an aspect of the disclosure

FIG. 15 is a cross-sectional view of the end cap of FIG. 14, shown engaged with the near port connector;

FIG. 16 is a perspective view of an end cap useable with the near port connector of FIG. 2, in accordance with an aspect of the disclosure

FIG. 17 is a cross-sectional view of the end cap of FIG. 16, shown engaged with the near port connector;

FIG. 18 is a cross-sectional view of a near port connector included in the catheter assembly of FIG. 1, in accordance with an aspect of the disclosure;

FIG. 19 is a cross-sectional view of a near port connector included in the catheter assembly of FIG. 1, in accordance with an aspect of the disclosure;

FIG. 20A is a cross-sectional view of a near port connector included in the catheter assembly of FIG. 1, with a ball valve thereof in an open position, in accordance with an aspect of the disclosure;

FIG. 20B illustrates the near port connector of FIG. 20A with the ball valve thereof in a closed position, in accordance with an aspect of the disclosure;

FIG. 21A is a cross-sectional view of a near port connector included in the catheter assembly of FIG. 1, with a push-pull valve thereof in an open position, in accordance with an aspect of the disclosure;

FIG. 21B illustrates the near port connector of FIG. 21A with the push-pull valve thereof in a closed position, in accordance with an aspect of the disclosure;

FIG. 22 is a perspective view of a catheter assembly of FIG. 1, in accordance with an aspect of the disclosure; and

FIG. 23 is a perspective view of a catheter assembly of FIG. 1, in accordance with an aspect of the disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

As used in this specification, the words “proximal” and “distal” refer to the direction closer to and away from, respectively, a user who would place the device into contact with a patient. Thus, for example, the end of a device first touching the body of the patient would be the distal end, while the opposite end of the device (e.g., the end of the device being manipulated by the user) would be the proximal end of the device.

Spatial or directional terms, such as “left”, “right”, “inner”, “outer”, “above”, “below”, and the like, are not to be considered as limiting as the invention can assume various alternative orientations.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

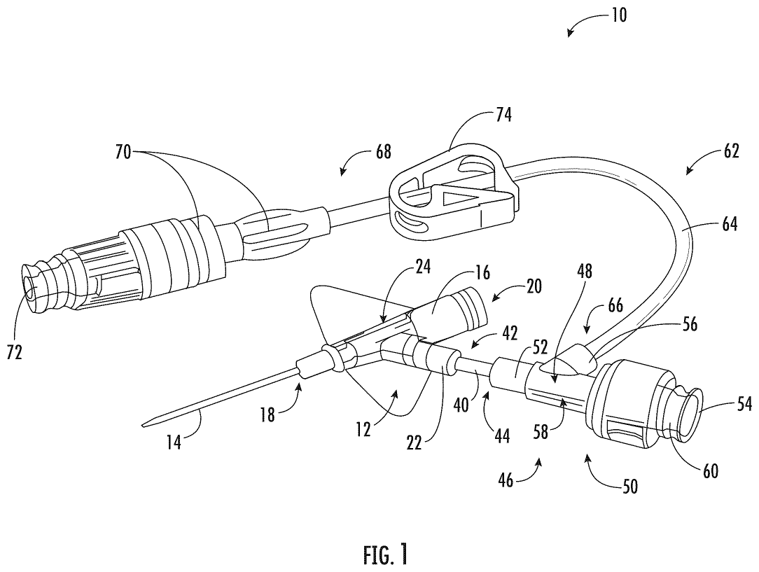

Referring to FIG. 1, shown is a non-limiting embodiment of a catheter assembly 10 for facilitating blood draw and/or administering of fluids for a patient. In the illustrated embodiment, the catheter assembly 10 is configured similar to a BD Nexiva™ closed peripheral IV catheter system, although it is recognized that the catheter assembly 10 may have another construction, according to additional aspects of the disclosure.

As shown in FIG. 1, catheter assembly 10 is constructed/arranged as an integrated catheter assembly that includes an integrated near-patient connector and extension set, as described in further detail below.

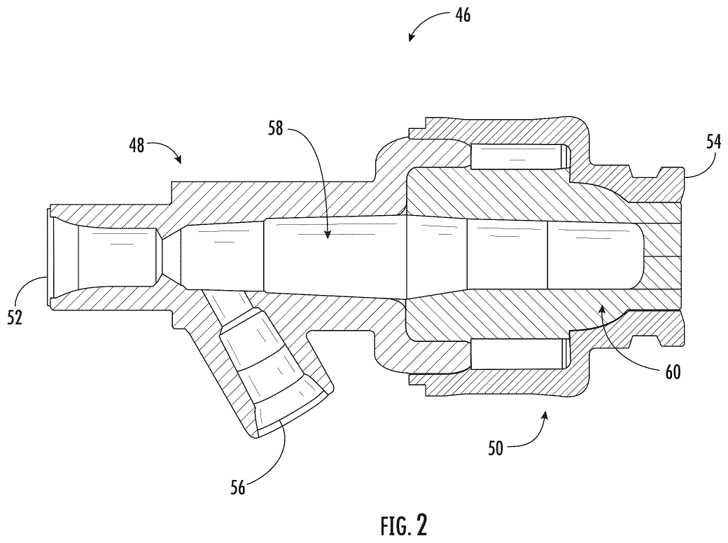

As shown in FIG. 2, the catheter assembly 10 includes a catheter adapter 12 and associated catheter 14. The catheter adapter 12 may include an adapter body 16 having a distal adapter port or end 18 and a proximal adapter port or end 20, as well as an additional or “side” adapter port 22 that may disposed between the distal end 18 and the proximal end 20. The catheter adapter 12 may include a (first) lumen 24 extending through the distal end 18 and the proximal end 20. The catheter 14 extends from the distal end 18 of catheter adapter 12 and may be configured as a PIVC that is placed into a vein of the patient, with a distal end or tip of the catheter 14 positioned appropriately within the vein to enable a blood draw from the patient. Catheter 14 may be formed of any suitable material and may be of any useful length, as known to those of skill in the art.

While not shown in FIG. 1, it is recognized that catheter assembly 10 may further include a needle subassembly provided at the proximal end 20 of the catheter adapter 12 for introducing the catheter 14 into the patient. The needle subassembly would include a needle that extends through the catheter adapter 12 (i.e., through lumen 24 thereof) and into the catheter 14, in a catheter-over-needle arrangement, for example. The needle subassembly may also comprise a body that includes a connector portion and a wing, with the connector portion engaging the proximal end 20 of the catheter adapter 12 and the wing configured to flex or deflect in order to control movement of the needle relative to the catheter 14 and/or catheter adapter 12, including during insertion of the needle into the vasculature of a patient and during a subsequent withdrawal of the needle out from the catheter 14 and out through the catheter adapter 12.

Referring still to FIG. 1, in some non-limiting embodiments or aspects, the catheter assembly 10 may include a first fluid conduit 40 extending from the side port 22, with the first fluid conduit 40 integrated with the side port 22. First fluid conduit 40 may be formed of any suitable material known to those of skill in the art and may have a distal end 42 and a proximal end 44. The distal end 42 of first fluid conduit 40 is coupled to port 22, while the proximal end 44 of first fluid conduit 40 may be coupled to a near-patient or “near port” connector 46 (hereafter “connector 46”). In some embodiments, the near port connector 46 may be integrated with first fluid conduit 40, while in other embodiments the near port connector 46 may be provided as a separate component that can be selectively attached and detached from the first fluid conduit 40.

As shown in FIG. 1 and in more detail in FIG. 2, according to an exemplary aspect of the disclosure, connector 46 includes first or distal housing portion 48 and a second or proximal housing portion 50. The housing portion 48 includes a distal connector end or port 52 thereon, while the housing portion 50 includes a proximal connector end or port 54 thereon. The housing portion 48 also includes a side connector port 56 thereon that is disposed between the distal and proximal ports 52, 54. According to embodiments, the connector 46 may be configured as a t-connector (e.g., one side port arranged at a 90 degree angle relative to a longitudinal axis of connector 46) or a y-connector (e.g., one side port arranged at a 15-85 degree angle relative to a longitudinal axis of connector 46). The connector 46 includes a (second) lumen 58 therein that is defined by the housing portions 48, 50 and the ports 52, 54, 56, with the lumen 58 having any number of branches suitable for the type of connector, such as a branch extending between distal and proximal ports 52, 54 and a branch provided to the side port 56.

In accordance with some embodiments or aspects, a needleless access connector 60, which may be configured as a split-septum valve (hereafter “split-septum valve 60”), is provided in the proximal port 54 of connector 46. The split-septum valve 60 may be retained within connector 46 upon coupling of the distal housing portion 48 to the proximal housing portion 50, with the split-septum valve 60 providing a near-patient access port to the catheter assembly 10 via which external devices may be connected to catheter assembly 10. In one non-limiting example, a blood draw device such as the BD PIVO™ may be connected to catheter assembly via connector 46 and the split-septum valve 60 thereof.

In some non-limiting embodiments or aspects, catheter assembly 10 may also include an extension set 62 coupled to the side port 56 of connector 46. The extension set 62 may include a second fluid conduit 64 having a distal end 66 and a proximal end 68. The distal end 66 of the second fluid conduit 64 may be coupled to side port 56 of connector 46, while the proximal end 68 of the second fluid conduit 64 may include a proximal connection or port 70—hereafter referred to as a “far port 70.” In some embodiments, the far port 70 may comprises a luer connection to which a fluid delivery device may be connected. In some embodiments, a needleless access connector 72 may be provided in/on the far port 70. A clamp 74 may be provided on the second fluid conduit 54, at a location between the ends 66, 68 thereof, that allows for occlusion thereof.

The extension set 62 may be used to provide a fluid path to or from the catheter assembly 10 to enable fluid or medication delivery, blood aspiration, or connection to an ex-vivo hemodynamic monitoring device that monitors blood pressure, heart rate, and/or pulse contour of a patient, as non-limiting examples. In some embodiments, a fluid delivery device may be coupled to the far port connector 70 to perform an intravenous infusion via a gravity flow drip, syringe push, or saline flush, as non-limiting examples.

According to aspects of the disclosure, it is desired to configure the near port connector 46 to be able to withstand increased pressure levels that might be experienced during use of the catheter assembly 10. For example, as described above, far port 70 may often be utilized for purposes of a fluid delivery in the catheter assembly 10, such as for performing saline flush where a saline solution is injected through the extension set 62, into side port 56 and out distally through the connector 46, and then through first fluid conduit 40 and into/through catheter adapter 12 and catheter 14. When performing such a flush, it is recognized that a backpressure may be applied against the split septum valve 60 of connector 46 and, in some instances, this backpressure may be significant. For example, if the indwelling catheter 14 is occluded when a flush is performed, the backpressure applied against the split septum valve 60 may be greatly increased (e.g., a backpressure exceeding 30 psi), and it is desirable for the connector 46 to be configured to be able to withstand high backpressure levels, in order to prevent leakage from the connector in a high pressure environment (i.e., a proximal flow of fluid through the split septum valve 60).

As described in further detail below, in some embodiments connector 46 may include one or more features incorporated therein that increase a backpressure capability of the connector, while in other embodiments a separate component may be coupled to the connector 46 to prevent leaks from the connector.

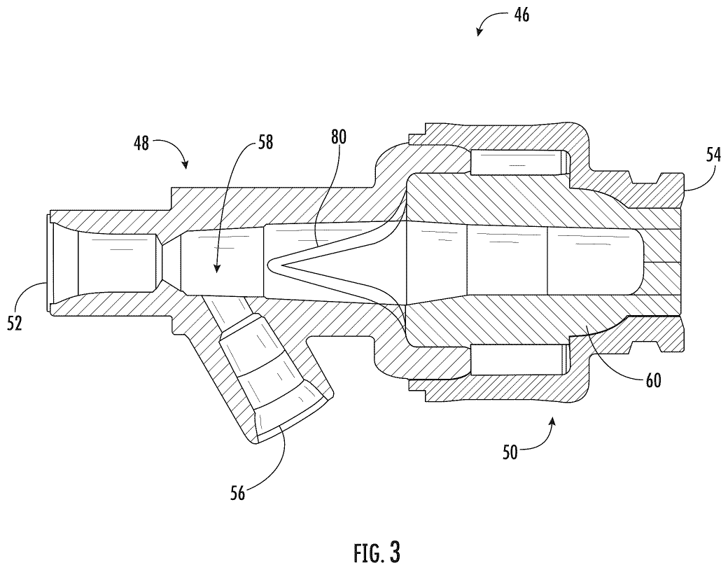

Referring now to FIGS. 3-5, embodiments of a near port connector 46 are shown that incorporate an additional valve therein to increase the backpressure capability of the connector 46, according to non-limiting aspects of the disclosure.

Referring first to FIG. 3, a near port connector 46 is illustrated that includes a duckbill valve 80 therein. The duckbill valve 80 is positioned within distal housing portion 48 (i.e., within lumen 58 distal housing portion 48). Specifically, the duckbill valve 80 is positioned within distal housing portion 48 at a location that is proximal to sideport 56 and distal to split septum valve 60. The duckbill valve 80 is configured to have a higher backpressure rating than the split septum valve 60, therefore increasing the backpressure rating of the connector 46. In some non-limiting embodiments, the duckbill valve 80 increases the backpressure rating of the connector 46 to over 30 psi, such as up to 60-80 psi and preferably up to 325 psi, in order to prevent a proximally-directed flow or leakage of fluid out from connector 46 at such backpressure.

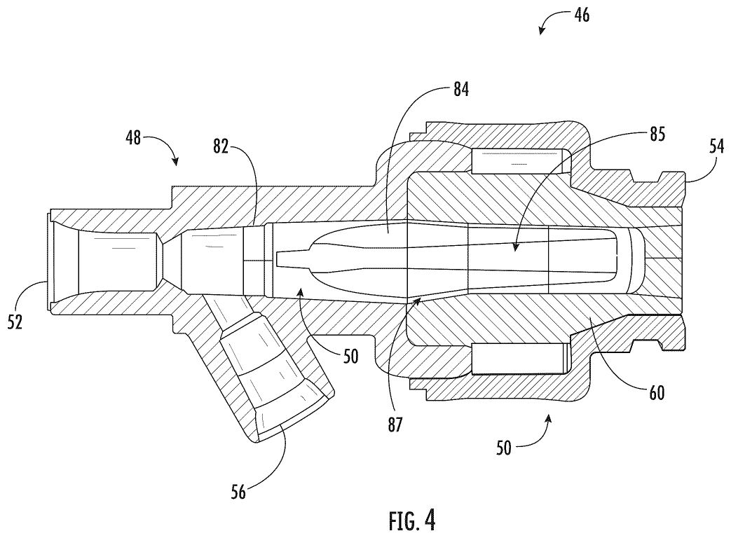

Referring next to FIG. 4, a near port connector 46 is illustrated that includes a secondary valve 82 therein, along with a valve actuator 84 that may be moved within connector 46 to open the secondary valve 82. The secondary valve 80 may, for example, comprise a disc-shaped valve having a septum formed therein, and may positioned within distal housing portion 48 (i.e., within lumen 58 distal housing portion 48) at a location that is proximal to sideport 56 and distal to split septum valve 60, with the secondary valve 82 having a higher backpressure rating than the split septum valve 60, so as to increase the backpressure rating of the connector 46. The valve actuator 84 may comprise an elongated member with a lengthwise channel 85 formed therethrough. The valve actuator 84 may be positioned partially within distal housing portion 48 (in lumen 58 thereof) and extend proximally back into proximal housing portion 50, with the valve actuator 84 seated within a cavity 87 defined in split-septum valve 60 (that forms another portion of lumen 58).

In use of connector 46, the secondary valve 80 is in a normally closed state, so as to provide increased backpressure capability to the connector 46, such as during flushing of the catheter assembly 10 via far port 66. The secondary valve 80 may be caused to open upon connection of an external device to the connector at proximal port 54. That is, valve actuator 84 may be pushed distally within lumen 58 upon connection of an external device to the connector at proximal port 54, with a male luer connector or blunt cannula pushing the valve actuator 84 distally until the valve actuator 84 engages/opens the secondary valve 80. With the external device subsequently disconnected from the connector 46, the valve actuator 84 may then return to its original position, either by a return spring or the tapered design of the actuator/secondary valve interface.

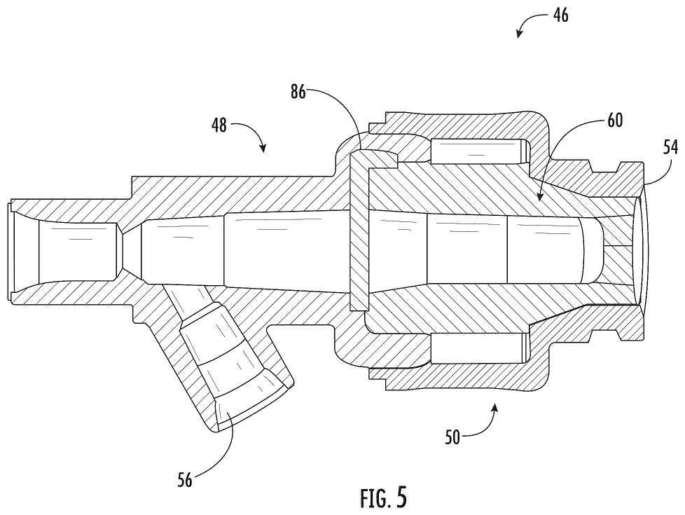

Referring next to FIG. 5, a near port connector 46 is illustrated that includes a swinging hinge valve 86 therein. The swinging hinge valve 86 is positioned within distal housing portion 48 (i.e., within lumen 58 distal housing portion 48) and is configured to be closed in a normal/non-actuated state. Specifically, the swinging hinge valve 86 is positioned within distal housing portion 48 at a location that is proximal to sideport 56 and distal to split septum valve 60. The swinging hinge valve 86 is configured to have a higher backpressure rating than the split septum valve 60, therefore increasing the backpressure rating of the connector 46. In some non-limiting embodiments, the swinging hinge valve 86 increases the backpressure rating of the connector 46 to over 30 psi, such as up to 60-80 psi and preferably up to 325 psi, in order to prevent a proximally-directed flow or leakage of fluid out from connector 46 at such backpressure. In use of connector 46, the swinging hinge valve 86 may be caused to open when a distally-directed pressure is applied through the proximal port 54 (e.g., a fluid pressure or mechanical pressure applied by coupling of a male connection of an external device), but otherwise seals off and holds backpressure against the body of the split-septum valve 60.

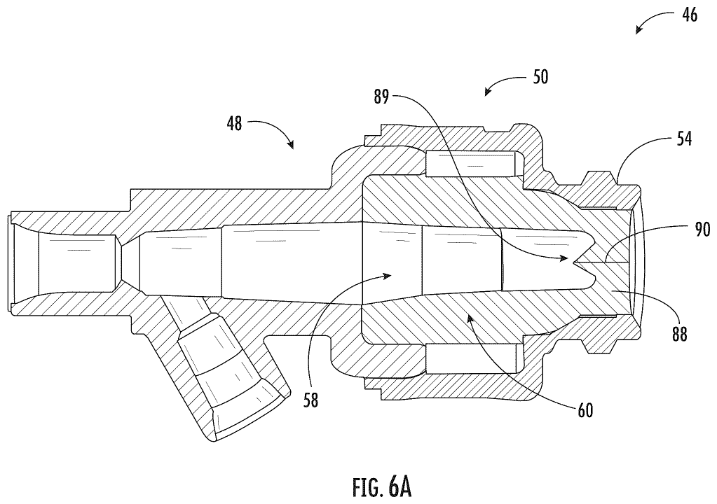

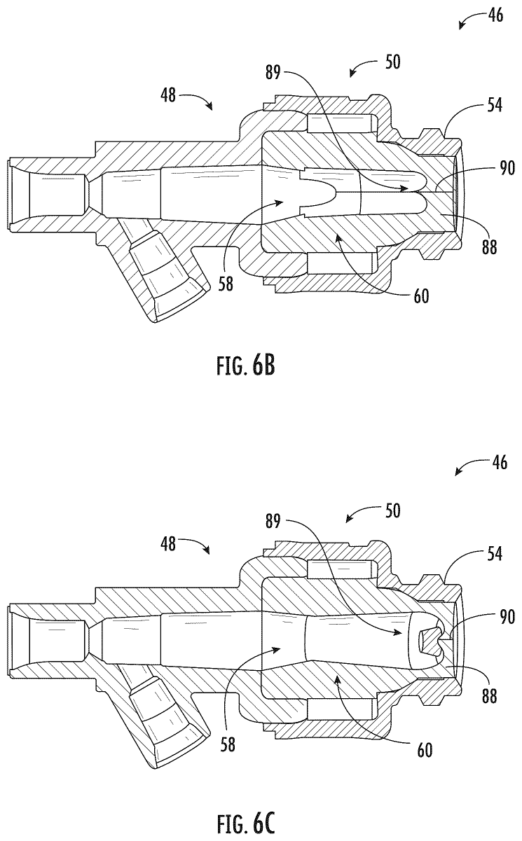

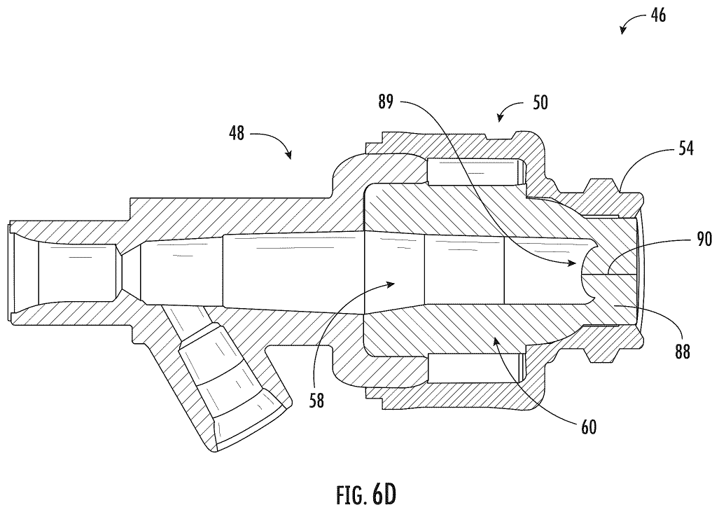

Referring now to FIGS. 6A-6D, embodiments of a near port connector 46 are shown that include a modified split-septum valve 60 that increases the backpressure capability of the connector 46, according to non-limiting aspects of the disclosure. That is, an underside (distal face) of the septum membrane 88 of the split-septum valve 60, is specifically contoured to increase the backpressure capability of the split-septum valve 60. In the embodiment of FIGS. 6A and 6B, the underside 89 of the septum membrane 88 has a duckbill configuration. In the embodiment of FIG. 6C, the underside 89 of the septum membrane 88 has a general duckbill-like configuration, with a frustoconical shaped protrusion extending out therefrom. In the embodiment of FIG. 6D, the underside 89 of the septum membrane 88 has a dome-shaped configuration. While specific shaping/contouring of the underside 89 of the septum membrane 88 is shown in FIGS. 6A-6D, it is recognized that the underside 89 of the septum membrane 88 may have other appropriate shapings/contourings, including a trapezoidal configuration or other geometries that may be symmetrical or asymmetrical to the slit in the septum membrane 88. In any appropriate configuration, the shaping/contouring of the underside 89 of the septum membrane 88 causes the slit 90 in the septum membrane 88 to push together when a backpressure is applied thereto. In some non-limiting embodiments, the shaping/contouring of the underside 89 of the septum membrane 88 increases the backpressure rating of the connector 46 to over 30 psi, such as up to such as up to 60-80 psi and preferably up to 325 psi, in order to prevent a proximally-directed flow or leakage of fluid out from connector 46 at such backpressure.

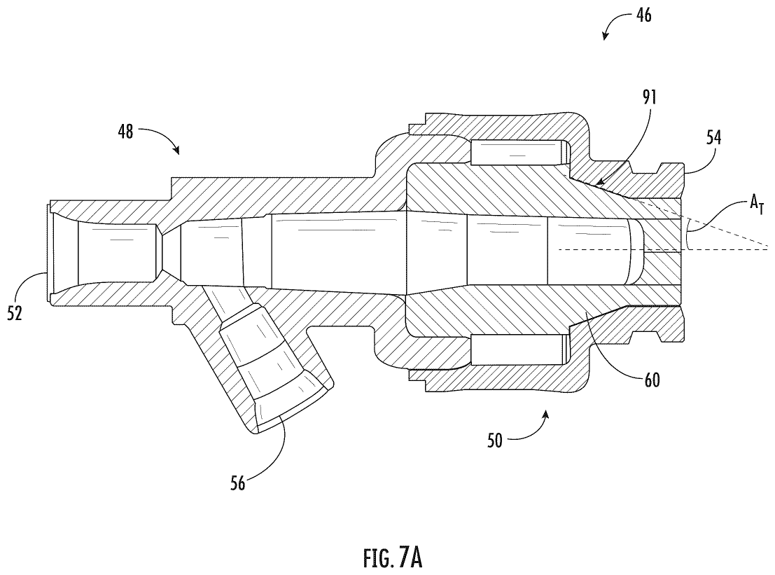

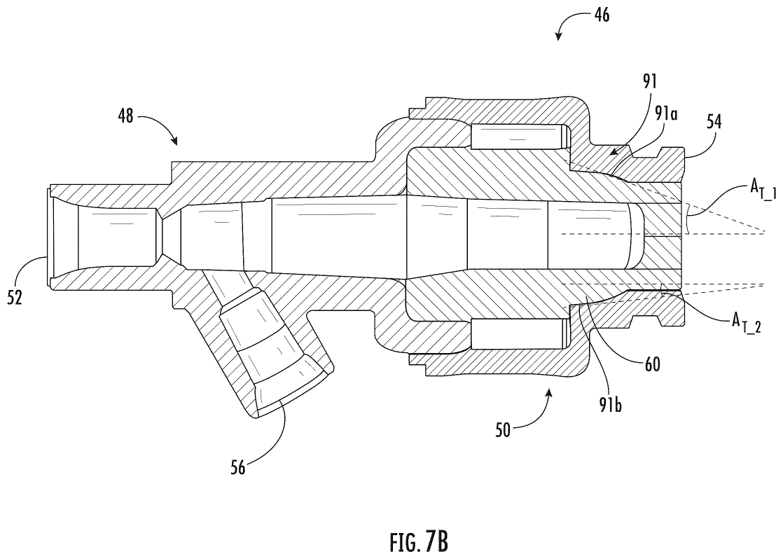

Referring now to FIGS. 7A and 7B, embodiments of a near port connector 46 are shown that provide a desired backpressure capability, while also being optimized such that the split-septum valve 60 opens uniformly and with decreased activation force when engaged by various types of connectors, including blunt cannulas and male luer connectors, as non-limiting examples. In particular, an inner surface of the proximal housing portion 50 is configured so that a taper angle, A_{T}, between a compressive zone 91 of the proximal housing portion 50 and the lower sidewalls of the split-septum valve 60 is within a specified range. According to an exemplary embodiment, the compressive zone 91 is configured to have a taper angle A_{T} of between 25 and 45 degrees, with such a taper angle enabling an earlier opening of the split-septum valve 60 (i.e., of the slit 90 in split-septum valve 60) when engaged by a connector, thereby decreasing a needed activation force and reducing the chances of split-septum valve 60 being damaged during valve activation. In some embodiments, and as shown in FIG. 7A, the compressive zone 91 may be configured to have a constant/singular taper angle A_{T} along a length thereof. In other embodiments, and as shown in FIG. 7B, the compressive zone 91 may be configured to include multiple segments 91a, 91b formed at different angles, with a taper angle A_{T_1} of the proximal-most segment 91a being larger than the taper angle A_{T_2} of the distal-most segment 91b, which allows for more uniform wall thickness to improve filling during injection molding.

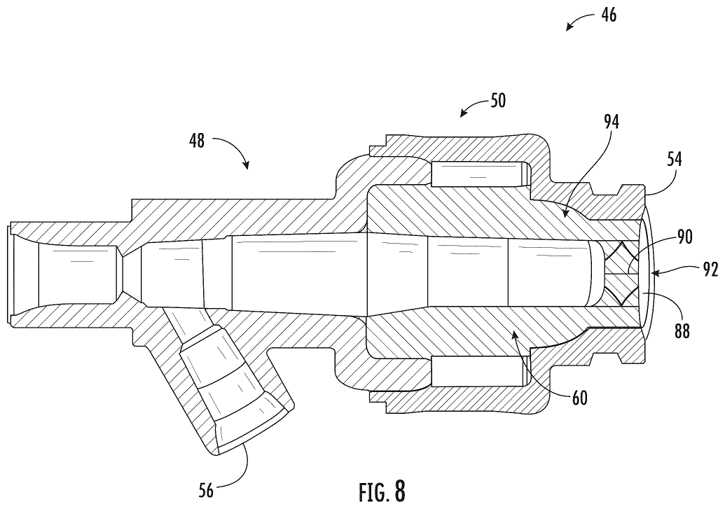

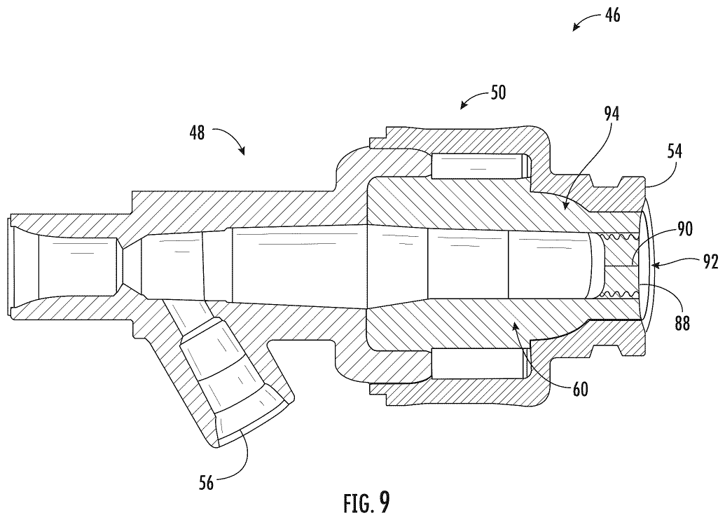

According to other non-limiting aspects of the disclosure, a near port connector 46 may be provided that includes a modified split-septum valve 60 that is formed (at least partially) of a stiffer material that increases the backpressure capability of the connector 46. In some embodiments, an entirety of the split-septum valve 60 may be formed of a material having a higher modulus of elasticity (i.e., a stiffer material grade) that decreases compliance in the system and therefore increases the backpressure performance of the split-septum valve 60. In other embodiments, the split-septum valve 60 may be formed as a bi-component/bi-material valve, where only a portion thereof (i.e., the septum membrane 88 including slit 90) is formed of a material having a higher modulus of elasticity (i.e., a stiffer material grade). In such a bi-component/bi-material embodiment, a first valve portion 92 and a second valve portion 94 may be joined together via cross-linking of material, melted together, or via a screw-in feature. FIGS. 8 and 9 provide two examples of a screw-in feature, with a stiffer first valve portion 92 (including at least the septum membrane 88) being screwed into a more compliant second valve portion 94. In some non-limiting embodiments, the forming of at least a portion of the split-septum valve 60 from a stiffer material increases the backpressure rating of the connector 46 to over 30 psi, such as up to such as up to 60-80 psi and preferably up to 325 psi, in order to prevent a proximally-directed flow or leakage of fluid out from connector 46 at such backpressure.

Referring now to FIGS. 10 and 11, embodiments of a near port connector 46 are shown that include a modified split-septum valve 60 and connector housing (distal housing portion 48 and proximal housing portion 50) that increases the backpressure capability of the connector 46, according to non-limiting aspects of the disclosure.

Referring first to FIG. 10, a near port connector 46 is illustrated that includes a split-septum valve 60 having a secondary seal 96 formed thereon, with the secondary seal 96 having a higher backpressure rating than a first seal 98 formed by the septum membrane 88. In some embodiments, the first seal 98 may thus be designed primarily to prevent microbial ingress, while the secondary seal 96 may be designed for a maximum backpressure rating (e.g., over 30 psi, and up to such as up to 60-80 psi and preferably up to 325 psi). As shown in FIG. 10, the secondary seal 96 is formed via a pair of protrusions 100 that extend inwardly to narrow the cavity 85 in the split-septum valve 60. The protrusions 100 are configured to deflect inwardly toward each other and come in contact with each other upon application of a radially inward-directed pressure thereof, such as when the distal housing portion 48 and proximal housing portion 50 are secured together (with the split-septum valve 60 housed therein), in order to provide the secondary seal 96. To provide for this deflection of the protrusions 100, the proximal housing portion 50 includes a reduced diameter portion 102 (formed by radially-inward extending bumps 104) that press inwardly against the area of the split-septum valve 60 including the secondary seal 96. In use of connector 46, when the split-septum valve 60 in engaged by a male connector of an external device, the split-septum valve 60 is caused to move downward, such that the secondary seal 96 is no longer compressed by the reduced diameter portion 102 of the proximal housing portion 50, such that the secondary seal 96 opens. In some embodiments, lube may be added to the interface between the proximal housing portion 50 and the split-septum valve 60 so that the split-septum valve 60 can slide more freely.

Referring next to FIG. 11, a near port connector 46 is illustrated that includes a split-septum valve 60 and proximal housing portion 50 having features thereon that prevent a proximally-directed movement of the split-septum valve 60. That is, a valve shelf 105 and a housing shelf 106 are added to the split-septum valve 60 and proximal housing portion 50, respectively, to increase a surface area that counteracts a backpressure applied to the connector 46 that might cause the split-septum valve 60 to pull out of the circumferentially compressed pocket (i.e., proximal port 54) of the proximal housing portion 50. In some embodiments, a second valve shelf 108 may be added to the split-septum valve 60 proximal to the valve shelf 105, that further increases the surface area contact that counteracts a backpressure applied to the connector 46 that might cause the split-septum valve 60 to pull out of the circumferentially compressed pocket (i.e., proximal port 54) of the proximal housing portion 50. With a proximal movement of the split-septum valve 60 relative to the proximal housing portion 50 prevented, the backpressure rating of the connector 46 may be increased to over 30 psi, such as up to such as up to 60-80 psi and preferably up to 325 psi, in order to prevent a proximally-directed flow or leakage of fluid out from connector 46 at such backpressure.

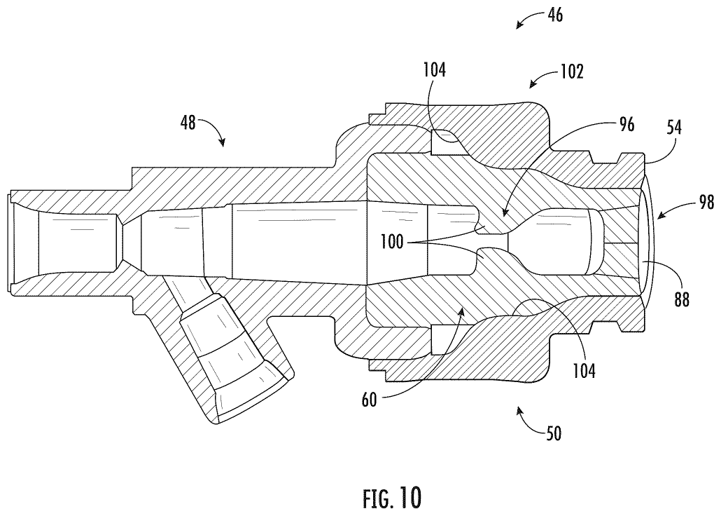

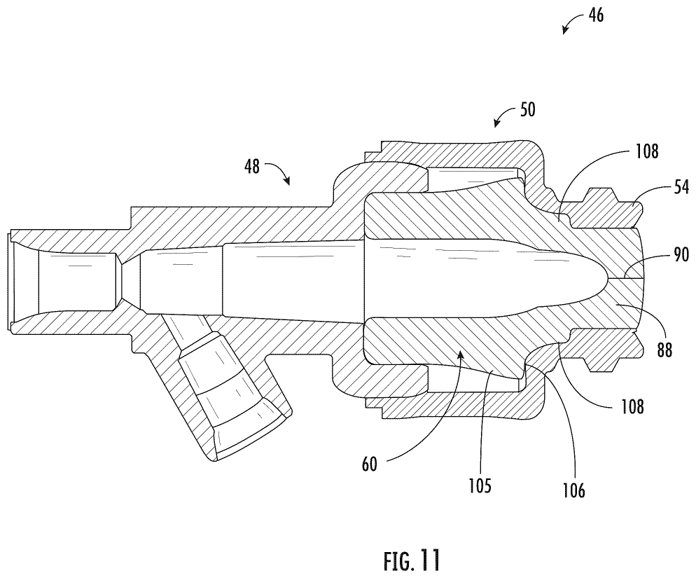

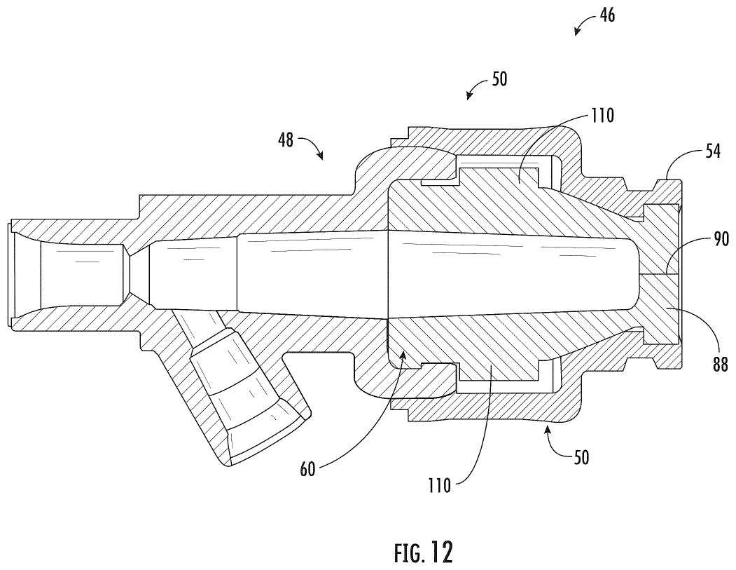

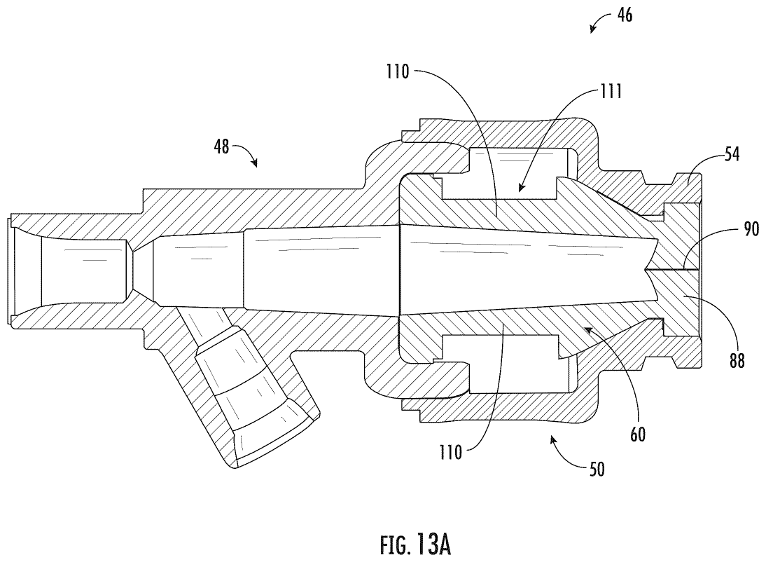

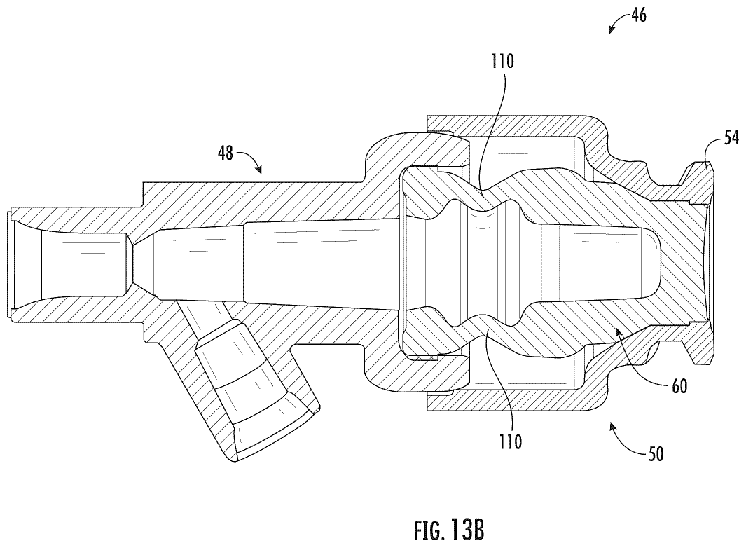

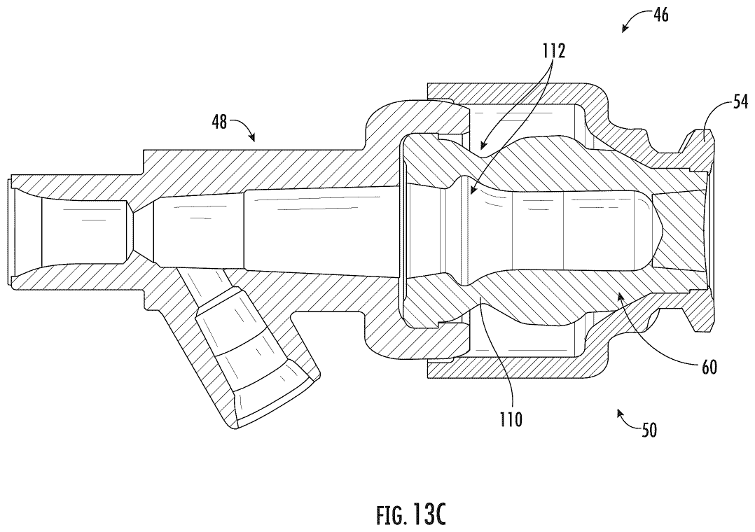

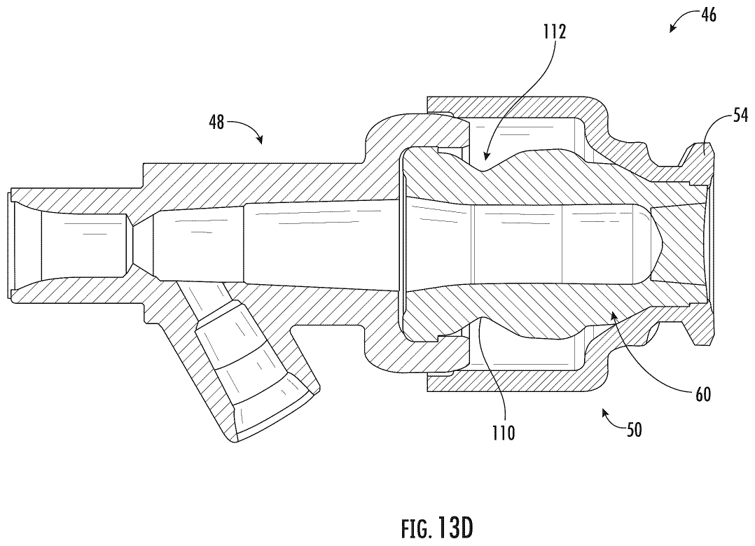

Referring now to FIG. 12 and FIGS. 13A-13D, embodiments of a near port connector 46 are shown that include a modified split-septum valve 60 that increases the backpressure capability of the connector 46, according to non-limiting aspects of the disclosure.

Referring first to FIG. 12, a near port connector 46 is illustrated that includes a split-septum valve 60 having sidewalls 110 with an increased thickness (as compared to existing split-septum valves in near port connectors). With the sidewalls 110 having an increased thickness, the stiffness of the split-septum valve 60 is increased—this due to there being a smaller gap between the sidewalls 110 and an interior surface of the proximal housing portion 50, and thus less room for the sidewalls 110 to deflect upon application of a backpressure to the split-septum valve 60. The increased stiffness of the split-septum valve 60 thus increases the backpressure rating of the connector 46 to over 30 psi, such as up to such as up to 60-80 psi and preferably up to 325 psi, in order to prevent a proximally-directed flow or leakage of fluid out from connector 46 at such backpressure.

Referring next to FIGS. 13A-13D, embodiments of a near port connector 46 are illustrated that include a split-septum valve 60 having sidewalls 110 with a decreased thickness (as compared to existing split-septum valves in near port connectors). According to embodiments, material may be removed from the sidewalls 110 according to any of a number of suitable patterns/profiles. Material may be removed from an inner surface, an outer surface, or both an inner surface and an outer surface of the sidewalls 110 in order to decrease a thickness thereof. FIGS. 13A-13D illustrate various non-limiting embodiments of exemplary near port connectors 46 having sidewalls 110 of reduced thickness, with FIG. 13A illustrating a split septum valve 60 having a rectangular cut-out 111 on the outer surface thereof, FIG. 13B illustrating a split septum valve 60 having an accordion-like profile with material removed from inner and outer surfaces of sidewalls 110, and FIGS. 13C and 13D illustrating a split septum valve 60 having curved cut-out(s) on inner and outer surfaces thereof (FIG. 13C) or on just the outer surface thereof (FIG. 13D).

With the sidewalls 110 having a decreased thickness, the compliance of the split-septum valve 60 in that area is also increased—with this increased compliance allowing the split-septum valve 60 additional space to absorb some of the backpressure applied thereto (i.e., the sidewalls 110 may deflect further outward) before the proximal end of the split-septum valve 60 (at septum membrane 88) is caused to move out of the compressive area of the proximal port 54 of the proximal housing portion 50. Accordingly, the connector 46 may exhibit an increased backpressure rating of over 30 psi, such as up to such as up to 60-80 psi and preferably up to 325 psi, in order to prevent a proximally-directed flow or leakage of fluid out from connector 46 at such backpressure.

In the embodiments of FIG. 12 and FIGS. 13A-13D, the septum membrane 88 may have a modified shape that alters compression of the slit 90 therein. For example, the septum membrane 88 may have an oval profile (rather than circular), in order to focus the compression on the slit 90 (i.e., long axis of the oval would be perpendicular to the slit). According to embodiments, the slit 90 in septum membrane 88 may be molded into the body of septum membrane 88 or may be formed (i.e., cut) into the body of septum membrane 88 post-molding.

While the embodiments of connector 46 shown and described in FIGS. 3-13 function to increase a backpressure rating of the connector, other embodiments of the disclosure may be directed to a connector 46 having one or more features (incorporated therein or attached thereto) that prevent leaks from the connector, such as by closing off a fluid path through the connector 46. Such components may generally referred to as a “fluid path shut-off” and function to prevent leaks from the connector 46 (through split-septum valve 60) in the event of application of a large backpressure thereto.

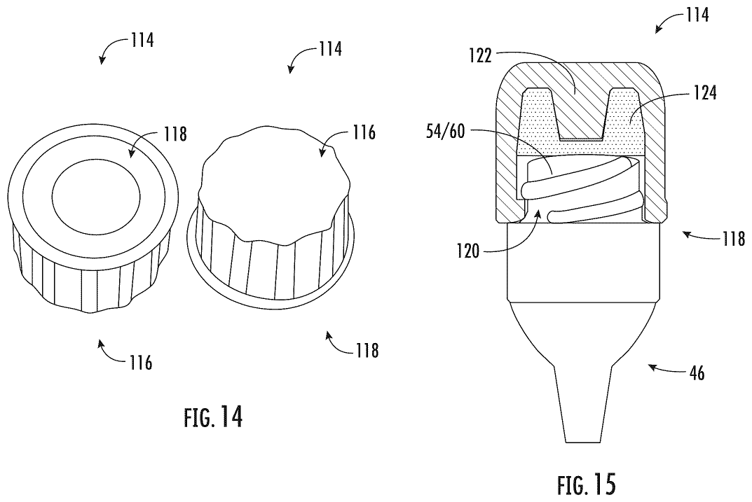

Referring to FIGS. 14 and 15, connector 46 is shown with an end cap 114 coupled thereto that prevents leaks from the connector 46 in the event of application of a large backpressure thereto, according to one embodiment of the disclosure. The end cap 114 may include a closed proximal end 116 and an open distal end 118, with the open distal end defining a cavity 120 in the end cap 114. A center protrusion 122 may extend into the cavity 120 from the closed proximal end 116. In some embodiments, the center protrusion 122 is configured to engage the split-septum valve 60 (i.e., penetrate through septum membrane 88), while in other embodiments the center protrusion 122 may be configured as “non-actuating”, i.e., does not engage the split-septum valve 60. An interior surface of the end cap 114 that defines the cavity 120 may comprise a threaded surface that engages the proximal port 54 of connector 46. A foam pad 124 may be retained in the cavity 120 and may contact the connector 46 (and split-septum valve 60), with the foam pad 124 retaining a disinfectant (e.g., isopropyl alcohol, iodine) therein. The foam pad 124 may be compressed upon engaging of the end cap 114 with connector 46, so as to seal and disinfect the split-septum valve 60.

When end cap 114 is connected to the connector 46, the end cap 114 functions to retain the split-septum valve 60 within connector 46 (i.e., prevent proximal movement within proximal housing portion 50) and retain the slit 90 of septum membrane 88 in a closed state. Accordingly, end cap 114 prevents a proximal flow of fluid through connector 46 and split-septum valve 60 when a large backpressure is applied thereto, thus preventing a fluid leak out from connector 46 during a fluid flush of the catheter assembly 10.

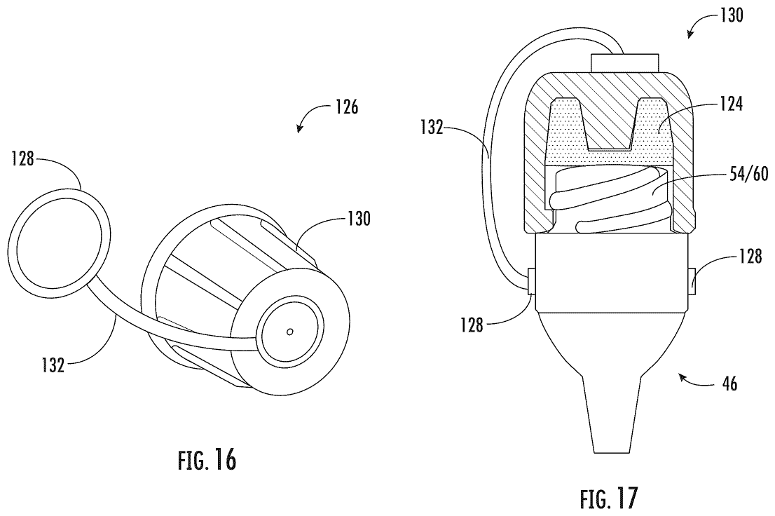

Referring to FIGS. 16 and 17, connector 46 is shown with an end cap 126 coupled thereto that prevents leaks from the connector 46 in the event of application of a large backpressure thereto, according to another embodiment of the disclosure. As compared to the end cap 114 of FIGS. 14 and 15, the end cap 126 is configured to be physically connected to the catheter assembly 10 using a tether. The end cap 126 thus includes a coupling loop 128 that may be secured about a component of catheter assembly 10 (e.g., connector 46 or catheter adapter 12), a cap member 130 that may be screwed onto proximal port 54, and a tether 132 that connects the coupling loop 128 and the cap member 130. According to embodiments, the cap member 130 of end cap 126 may be configured identical to the end cap 114 of FIGS. 14 and 15 (including the disinfecting foam pad 124). In other embodiments, the cap member 130 of end cap 126 may be a simple dead-end cap that screws onto proximal port 54. According to some embodiments, the end cap 126 may be initially covered in a protective wrapper, in order to provide a sterile component. According to some embodiments, a weak point could be included in the tether 132, so that a user could twist the tether 132 and break off the connection to remove the cap member 130 and dispose thereof, after use of the end cap 126.

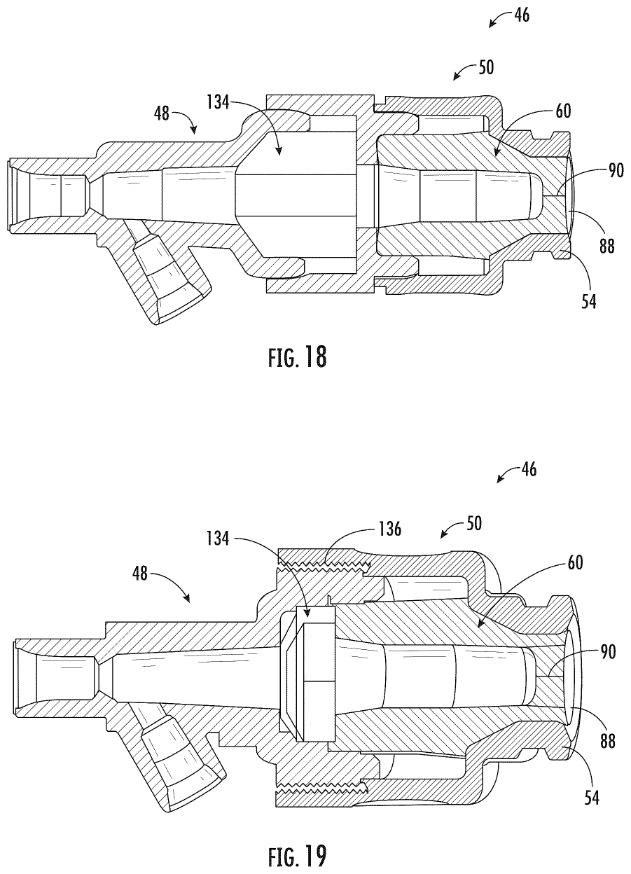

Referring to FIGS. 18 and 19, connector 46 is shown with an additional component (separate from split-septum valve 60) integrated therein that selectively opens/closes a fluid path through the connector 46. That is, connector 46 may include a Tuohy-Borst type valve 134 that is integrated into the distal housing portion 48 so as to be adjacent/under the split septum valve 60 (FIG. 18) or that is integrated into/with the split septum valve 60 (FIG. 19). The Tuohy-Borst valve 134 allows a user to isolate the split septum valve 60 from the rest of the fluid path (in catheter assembly 10) when the internal fluid backpressure might exceed the split septum valve's capabilities. In some embodiments, the Tuohy-Borst valve 134 may close the slit 90 of the split septum valve 60 by axially compressing the valve, such as by screwing the housing portions 48, 50 of connector 46 together via a locking collar 136 provided on the connector.

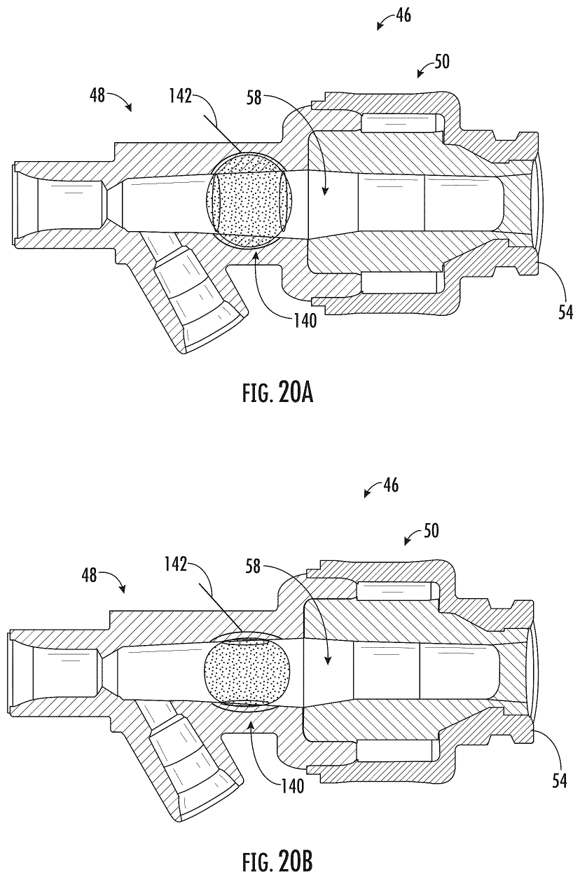

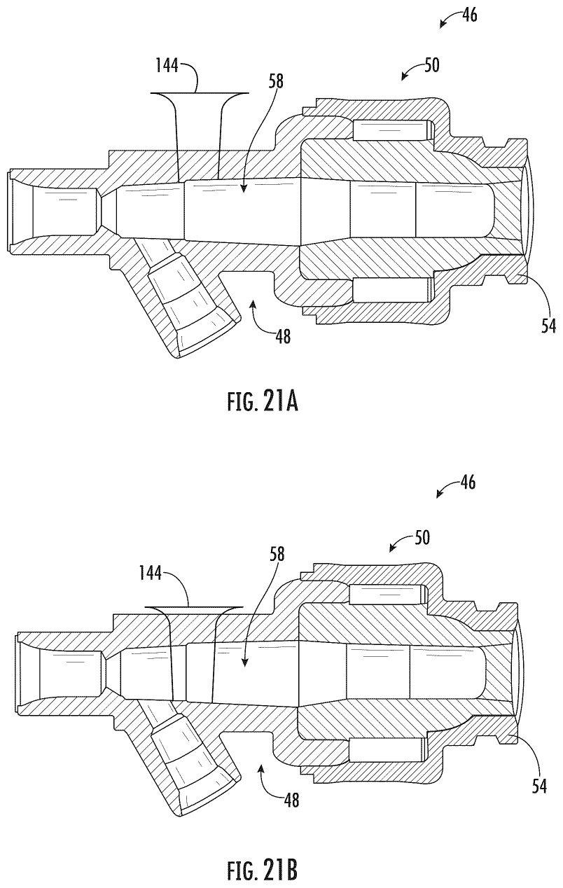

Referring to FIGS. 20A and 20B and FIGS. 21A and 21B, connector 46 may further include a separate valve component integrated therein (i.e., into distal housing portion 48) that may be controlled to selectively open/close a fluid path (i.e., lumen 58) through the connector 46.

Referring first to FIGS. 20A and 20B, a near port connector 46 is illustrated that includes a ball valve 140 integrated into the distal housing portion 48. The ball valve 140 may include a lever 142 thereon that extends to the exterior of the connector 46, with the lever 142 rotatable to selectively open/close a fluid path (i.e., lumen 58) through the connector 46. That is, the lever 142 may be rotated in a first direction to open the ball valve 140 and open the fluid path through the connector 46 (FIG. 20A) and may be rotated in a second direction to close the ball valve 140 and close the fluid path through the connector 46 (FIG. 20B).

Referring next to FIGS. 21A and 21B, a near port connector 46 is illustrated that includes a pull/push valve 144 integrated into the distal housing portion 48. The pull/push valve 144 may extend out from the connector 46 and may be “pulled” or “pushed” to selectively open/close a fluid path (i.e., lumen 58) through the connector 46. That is, when the pull/push valve 144 is in the “pulled” state (FIG. 21A), the valve is open and the fluid path through the connector 46 is open, and when the pull/push valve 144 is in the “pushed” state (FIG. 21B), the valve is closed and the fluid path through the connector 46 is closed. The pull/push valve 144 thus allows a user to isolate the near port connector 46 from the rest of the fluid path in catheter assembly 10 when the internal fluid pressure might exceed the backpressure capabilities of the connector 46. In some embodiments, and as shown in FIGS. 21A and 21B, the pull/push valve 144 is integrated into distal housing portion 48. In other embodiments not shown herein, the pull/push valve 144 is integrated into proximal housing portion 50—with the pull/push valve 144 then operating to compress the split-septum valve 60 inside the proximal port 54 to withstand the internal fluid pressures.

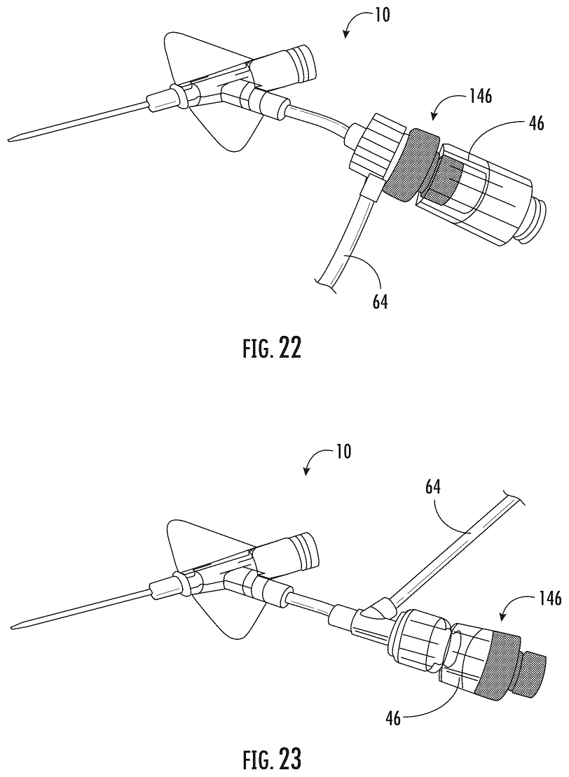

Referring to FIGS. 22 and 23, in still another embodiment, a separate in-line needle free connector, anti-reflux, or bi-direction valve, or interlink type connector (accessible with a blunt plastic cannula), which is generally indicated at 146, is added/connected to connector 46 to provide an additional layer of pressure capability. The separate in-line needle free connector 146 may be positioned distally (FIG. 22) or proximally (FIG. 23) of the primary connector 46, but—in either embodiment—the separate in-line needle free connector 146 is positioned proximal to a connection point/port of the second fluid conduit 64.

Beneficially, embodiments of the disclosure thus provide a near port connector for a catheter assembly that provides an improved back-pressure capability and/or leakage prevention. In some embodiments, the near port connector may include one or more pressure resistance-increasing features therein that are configured to increase a pressure capability of the split septum valve therein. In other embodiments, a separate cap or valve may be added/attached to the near port connector to selectively open/close a fluid path therethrough, so as to prevent leaks from the connector during a high backpressure event/occurrence.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A catheter assembly comprising:
a catheter comprising a catheter distal end and a catheter proximal end;
a catheter adapter comprising an adapter body including a distal adapter port, a proximal adapter port, and a side adapter port, the adapter body defining a lumen therein extending between the distal adapter port and the proximal adapter port, wherein the catheter proximal end is secured to the distal adapter port, and wherein the side adapter port is in fluid communication with the lumen;
a first fluid conduit comprising a distal end and a proximal end, the distal end of the first fluid conduit connected to the proximal adapter port;
a near port connector comprising a distal connector port, a proximal connector port, and a side connector port, the distal connector port connected to the proximal end of the first fluid conduit, with the near port connector comprising a split-septum valve provided in the proximal connector port; and
an extension set coupled to the side connector port, the extension set comprising:
a second fluid conduit comprising a distal end and a proximal end, the distal end coupled to the side connector port; and
a far port connector provided at the proximal end of the second fluid conduit;
wherein the near port connector is configured to withstand a proximally-directed fluid backpressure of greater than 30 pounds-per-square inch (psi), so as to prevent fluid leaks out from the near port connector during a fluid injection through the far port connector.

2. The catheter assembly of claim 1, wherein the near port connector comprises a duckbill valve positioned within a lumen of the near port connector, proximal to the side connector port and distal to the split-septum valve, the duckbill valve having a higher backpressure rating than the split-septum valve.

3. The catheter assembly of claim 1, wherein the near port connector comprises:
a secondary valve positioned within a lumen of the near port connector, proximal to the side connector port and distal to the split-septum valve, the secondary valve having a higher backpressure rating than the split-septum valve; and
a valve actuator element positioned within a lumen of the near port connector and proximally from the secondary valve, the valve actuator element including a channel formed therethrough;
wherein the valve actuator element is moveable distally within the lumen responsive to introduction of a male connector into the split-septum valve, with the valve actuator element opening the secondary valve upon such distal movement.

4. The catheter assembly of claim 1, wherein the near port connector comprises a swinging hinge valve positioned within a lumen of the near port connector, proximal to the side connector port and distal to the split-septum valve, the swinging hinge valve having a higher backpressure rating than the split-septum valve.

5. The catheter assembly of claim 1, wherein the split-septum valve comprises:
a septum membrane having a slit formed therethrough; and
a sidewall extending distally from the septum membrane, with the septum membrane and sidewall defining a cavity in the split-septum valve.

6. The catheter assembly of claim 5, wherein an underside of the septum membrane has a duckbill shape.

7. The catheter assembly of claim 5, wherein an underside of the septum membrane has a dome shape.

8. The catheter assembly of claim 5, wherein the split-septum valve comprises a bi-material component, with the sidewall formed of a first material and the septum membrane formed of a second material that has a higher modulus of elasticity than the first material.

9. The catheter assembly of claim 5, wherein the near port connector comprises a distal housing portion and a proximal housing portion that engage each other to secure the split-septum valve within the near port connector.

10. The catheter assembly of claim 9, wherein the sidewall comprises a pair of protrusions on opposing sides thereof, with the pair of protrusions extending radially inward into the cavity to narrow the cavity, with the pair of protrusions configured to deflect inwardly toward each other and come in contact with each other upon application of a radially inward-directed pressure thereto, thereby forming a secondary seal in the split-septum valve having a higher backpressure rating than the septum membrane.

11. The catheter assembly of claim 10, wherein the proximal housing portion includes a reduced diameter portion formed by a pair of radially-inward extending bumps, with the pair of radially-inward extending bumps pressing inwardly against the split-septum valve in an area of the pair of protrusions when the distal housing portion is secured to the proximal housing portion, thereby deflecting the pair of protrusions to form the secondary seal.

12. The catheter assembly of claim 9, wherein the proximal housing portion comprises a housing shelf and the sidewall comprises a valve shelf, with the housing shelf engaging with the valve shelf to increase a contact surface area between the split-septum valve and the proximal housing portion, in order to prevent proximal movement of the split-septum valve within the proximal housing portion responsive to a proximally-directed fluid backpressure.

13. The catheter assembly of claim 9, wherein a thickness of the sidewall is increased or decreased, so as to decrease or increase a compliance of the split-septum valve, respectively, so as to increase the backpressure rating of the split-septum valve.

14. The catheter assembly of claim 13, wherein the thickness of the sidewall is decreased, with the sidewall of the split septum valve having a rectangular cut-out on an outer surface thereof, a curved cut-out on the outer surface thereof, curved cut-outs on inner and outer surfaces thereof, or an accordion-like profile with material removed from inner and outer surfaces thereof.

15. The catheter assembly of claim 9, wherein the proximal housing portion comprises an inner surface defining a compressive zone that engages the split-septum valve to maintain the split-septum valve in a closed configuration, and wherein the inner surface in the compressive zone has a taper angle of between 25 and 45 degrees.

16. The catheter assembly of claim 15, wherein the inner surface in the compressive zone comprises a proximal-most segment and a distal-most segment having different taper angles, with the taper angle of the proximal-most segment being larger than the taper angle of the distal-most segment.

17. The catheter assembly of claim 1, wherein the near port connector is configured to withstand a proximally-directed fluid backpressure of up to 60-80 psi, and preferably up to 325 psi.

18. A catheter assembly comprising:
a catheter comprising a catheter distal end and a catheter proximal end;
a catheter adapter comprising an adapter body including a distal adapter port, a proximal adapter port, and a side adapter port, the adapter body defining a lumen therein extending between the distal adapter port and the proximal adapter port, wherein the catheter proximal end is secured to the distal adapter port, and wherein the side adapter port is in fluid communication with the lumen;
a first fluid conduit comprising a distal end and a proximal end, the distal end of the first fluid conduit connected to the proximal adapter port;
a near port connector comprising a distal connector port, a proximal connector port, and a side connector port, the distal connector port connected to the proximal end of the first fluid conduit, with the near port connector comprising a split-septum valve provided in the proximal connector port;
an extension set coupled to the side connector port, the extension set comprising:
a second fluid conduit comprising a distal end and a proximal end, the distal end coupled to the side connector port; and
a far port connector provided at the proximal end of the second fluid conduit; and
a fluid path shut-off attached to or integrated with the near port connector, to selectively close a fluid path through the near port connector and the split-septum valve, so as to prevent fluid leaks out from the near port connector during a fluid injection through the far port connector.

19. The catheter assembly of claim 18, wherein the fluid path shut-off comprises an end cap coupled to the proximal connector port, with the end cap sealing off the split-septum valve.

20. The catheter assembly of claim 19, wherein the end cap comprises a disinfecting foam pad retained within a cavity thereof, with the foam pad being compressed upon engaging of the end cap with the proximal connector port, so as to seal and disinfect the split-septum valve.

21. The catheter assembly of claim 19, wherein the end cap comprises a tethered end cap including:
a cap member that couples to the proximal connector port to seal off the split-septum valve;
a coupling loop configured to engage with the near port connector; and
a tether connected the cap member and the coupling loop.

22. The catheter assembly of claim 18, wherein the fluid path shut-off comprises a Tuohy-Borst valve that is integrated into the near port connector so as to be adjacent the split septum valve or integrated with the split septum valve, the Tuohy-Borst valve operable to close the fluid path through the near port connector and the split-septum valve.

23. The catheter assembly of claim 18, wherein the fluid path shut-off comprises a ball valve that is integrated into the near port connector so as to be distal to the split septum valve and proximal from the side connector port, the ball valve operable to close the fluid path through the near port connector and the split-septum valve.

24. The catheter assembly of claim 18, wherein the fluid path shut-off comprises a push-pull valve that is integrated into the near port connector so as to be distal to the split septum valve and proximal from the side connector port, the push-pull valve operable to close the fluid path through the near port connector and the split-septum valve.

25. The catheter assembly of claim 18, wherein the fluid path shut-off comprises one of an in-line needle free connector, an anti-reflux or bi-direction valve, or an interlink type connector, that is coupled to the near port connector at either the distal connector port or the proximal connector port.
